(19) United States
(12) Patent Application Publication
(10) Pub. No.: US 2014/0270070 A1
(43) Pub. Date: Sep. 18, 2014

(54) X-RAY RECORDING SYSTEM FOR DIFFERENTIAL PHASE CONTRAST IMAGING OF AN EXAMINATION OBJECT BY WAY OF PHASE STEPPING

(71) Applicant: SIEMENS AKTIENGESELLSCHAFT, Munich (DE)

(72) Inventor: Martin SPAHN, Erlangen (DE)

(21) Appl. No.: 14/191,680

(22) Filed: Feb. 27, 2014

(30) Foreign Application Priority Data
Mar. 15, 2013 (DE) 102013204604.9

(51) Int. Cl. A61B 6/00

(52) U.S. Cl. CPC A61B 6/484
USPC 378/62

(57) ABSTRACT

An x-ray recording system is for differential phase contrast imaging of an examination object via phase stepping. In an embodiment, the x-ray recording system includes at least one x-ray emitter for generating quasi coherent x-ray radiation; an x-ray image detector with pixels arranged in a matrix; a defraction or phase grating arranged between the examination object and the x-ray image detector; and an analyzer grating assigned to the phase grating, wherein x-ray emitter, x-ray image detector, phase grating and analyzer grating for the phase contrast imaging form components in an arrangement. According to an embodiment, at least one measuring apparatus for determining deviations in the geometric ratios of the components relative to one another from the geometry target, an analysis unit for evaluating the measured deviations, a computing unit for determining correction values and at least one correction device for setting the geometric ratios of the components are included.

### PRIORITY STATEMENT

The present application hereby claims priority under 35 U.S.C. §119 to German patent application number DE 102013204604.9 filed Mar. 15, 2013, the entire contents of which are hereby incorporated herein by reference.

### FIELD

At least one embodiment of the invention generally relates to an x-ray recording system for differential phase contrast imaging of an examination object by way of phase stepping having at least one x-ray emitter for the generation of quasi coherent x-ray radiation, an x-ray image detector with pixels arranged in a matrix, a defraction or phase grating, which is arranged between the examination object and the x-ray image detector, and an analyzer grating assigned to the phase grating, wherein x-ray emitter, x-ray image detector, phase grating and analyzer grating for the phase contrast imaging form critical components in a predetermined arrangement.

### BACKGROUND

Differential phase contrast imaging represents an imaging method which has received a great deal of attention for some time particularly in the Talbot Lau interferometer arrangement. For instance, the publication by F. Pfeiffer et al. [1], “Hard X-ray dark-field imaging using a grating interferometer”, Nature Materials 7, pages 134 to 137 describes that the use of x-ray-optical gratings on the one hand allows for the recording of x-ray images in the phase contrast, which deliver additional information relating to an examination object. On the other hand, there is also the option of using not only the phase information but also the amplitude information of scattered radiation for imaging purposes. Imaging can be generated in this way, which is exclusively based on the scattered portions of the x-ray radiation defracted by the examination object, in other words a minimal angle scattering. Very minor density differences in the examination object can herewith be shown using high resolution. The same can also be inferred from Joseph J. Zambelli, et al. [2], “Radiation dose efficiency comparison between differential phase contrast CT and conventional absorption CT”, Med. Phys. 37 (2010), pages 2473 to 2479.

The wave nature of particles such as x-ray quanta allows for the description of phenomena such as refraction and reflection with the aid of the complex refraction index

n=1−δ+iβ.

In such cases the imaginary part β describes the absorption, such as that which underlies current clinical x-ray imaging, e.g. computed tomography, angiography, radiography, fluoroscopy or mammography, and the real part δ describes the phase shift, which is taken into consideration during the differential phase imaging.

DE 10 2010 018 715 A1 describes an x-ray recording system, with which an x-ray recording system for phase contrast imaging of an examination object is used for high-quality x-ray imaging, said x-ray recording system having at least one x-ray emitter with a plurality of field emission x-ray sources for emitting a coherent x-ray radiation, an x-ray image detector, a defraction grating arranged between the examination object and the x-ray image detector G₁ and a further grating G₂, which is arranged between the defraction grating G₁ and the x-ray image detector.

An x-ray image recording system, with which a differential phase contrast imaging of the type cited in the introduction can be implemented, is known for instance from U.S. Pat. No. 7,500,784 B2, which is explained with the aid of FIG. 1.

FIG. 1 shows the typical essential features of an x-ray recording system for an interventional suite with a C-arm 2 held by a stand 1 in the form of a six-axis industrial or articulated arm robot, at whose ends an x-ray radiation source, for instance an x-ray emitter 3 with x-ray tube and collimator, and an x-ray image detector 4 as image recording unit are attached.

By way of the articulated arm robot known for example from U.S. Pat. No. 7,500,784 B2, which preferably has six axes of rotation and thus six degrees of freedom, the C-arm 2 can be displaced spatially as required, by being rotated for example about a center of rotation between the x-ray emitter 3 and the x-ray image detector 4. The angiographic x-ray system 1 to 4 according to the invention can be rotated in particular about centers of rotation and axes of rotation in the C-arm plane of the x-ray image detector 4, preferably about the center point of the x-ray image detector 4 and about axes of rotation intersecting the center point of the x-ray image detector 4.

The known articulated arm robot has a basic frame, which is fixedly mounted on a floor for instance. A carousel is rotatably fastened thereto about a first axis of rotation. A robot swing arm is pivotably attached to the carousel about a second axis of rotation, to which a robot arm is fastened rotatably about a third axis of rotation. A robot hand is rotatably attached about a fourth axis of rotation at the end of the robot arm. The robot hand has a fastening element for the C-arm 2, which can be pivoted about a fifth axis of rotation and can be rotated about a sixth axis of rotation which runs at right angles thereto.

The realization of the x-ray diagnostics facility is not dependent on the industrial robot. Conventional C-arm devices can also be used.

The x-ray image detector 4 can be a rectangular or square, flat semiconductor detector that is preferably made of amorphous silicon (a-Si). Integrating and possibly counting CMOS detectors can however also be used.

A patient 6 to be examined, as an examination object, is disposed in the radiation path of the x-ray emitter 3 on a table top 5 of a patient support couch. A system control unit 7 with an imaging system 8 is connected to the x-ray diagnostics facility, said imaging system 8 receiving and processing the image signals of the x-ray image detector 4 (control elements are not shown for instance). The x-ray images can then be viewed on displays of a monitor rack 9. The monitor lighting system 9 can be held by way of a ceiling-mounted, longitudinally-movable, pivotable, rotatable and height-adjustable carrier system 10 having a cantilever and lowerable support arm.

Instead of the x-ray system shown for instance in FIG. 1 with the stand 1 in the form of the six-axis industrial or articulated arm robot, as shown in simplified form in FIG. 2, the angiographic x-ray system can also have a normal ceiling or floor-mounted holder for the C-arm 2.

Instead of the C-arm 2 shown by way of example, the angiographic x-ray system can also have separate ceiling and/or floor-mounted holders for the x-ray emitter 3 and the x-ray image detector 4, which are fixedly electronically coupled for instance.

In currently highlighted arrangements for clinical phase contrast imaging, conventional x-ray tubes, currently available x-ray image detectors, such as are described for instance by Martin Spahn [3] in “Flachbilddetektoren in der Röntgendiagnostik”, Der Radiologe, [Flat image detectors in x-ray diagnostics, The Radiologist] Volume 43 (5-2003), pages 340 to 350, and three gratings G₀, G₁ and G₂ are used, such as is explained in closer detail with the aid of FIG. 2, which indicates a schematic structure of a Talbot Lau interferometer for the differential phase contrast imaging with extended tube focus, gratings G₀, G₁ and G₂ and pixelated x-ray image detector.

The x-ray beams 12 originating from a tube focus 11 of the non-coherent x-ray emitter R penetrate an absorption grating 13 (G₀) for the generation of coherent radiation, which brings about the local coherence of the x-ray radiation source, and an examination object 14, for instance the patient 6. By way of the examination object 14, the wave front of the x-ray beams 12 is deflected by the phase shift, as the normals 15 of the wave front without phase shift, i.e. without object, and the normals 16 of the wave front with phase shift clarify. The phase-shifted wave front then passes through a defraction or phase grating 17 (G₁) with a grating constant adjusted to the typical energy of the x-ray spectrum in order to generate interference lines and in turn an absorbing analyzer grating 18 (G₂) so as to read out the generated interference pattern. The grating constant of the analyzer grating 18 is adjusted to that of the phase grating 17 and the remaining geometry of the arrangement. The analyzer grating 18 is arranged for instance at the first or n-th Talbot distance. In such cases, the analyzer grating 18 converts the interference pattern into an intensity pattern, which can be measured by the detector. Typical grating constants for clinical applications lie at a few μm, such as can also be inferred for instance from the cited citations [1, 2].

If the tube focus 11 of the radiation source is sufficiently small and the generated radiation output is sufficiently large, it is possible to dispense with the first grating G₀, the absorption grating 13, such as is given if a plurality of field emission x-ray sources are provided as x-ray emitter 3 for instance, such as is known from DE 10 2010 018 715 A1 described below.

The differential phase shift is now determined for each pixel of the x-ray image detector 4 in that by way of a so-called “phase stepping” 19, which is indicated by an arrow, the analyzer grating 18 (G₂) is shifted in a number of steps (k=1, K, with e.g. K=4 to 8), about a corresponding fraction of the grating constants at right angles to the radiation direction of the x-ray beams 12 and laterally with respect to the arrangement of the grating structure, and the signal Sₖ developing for this configuration during the recording is measured in the pixel of the x-ray image detector 4 and thus the developed interference pattern is scanned. The parameters of a function describing the modulation (e.g. sinus function) are then determined for each pixel by a suitable fitting method, an adjustment or compensation method, to the thus measured signals Sₖ. The visibility, i.e. the standardized difference between the maximum and minimum signal (or in more precise terms: amplitude standardized to the average signal), is in such cases a measure of the characterization of the quality of a Talbot Lau interferometer. It is defined as a contrast of the scanned modulation.

$$V = \frac{{I}_{ma x} - {I}_{m i n}}{{I}_{m ax} + {I}_{m i n}} = \frac{A}{\overline{I}}$$

Furthermore, this equation A refers to the amplitude and Ī the average intensity. The visibility can assume values between zero and one, since all variables are positive and Iₘₐₓ>Iₘᵢₙ. Iₘᵢₙ>0 also applies in a real interferometer, so that the value range of V is expediently exhausted. Minimal intensities of greater than zero and all non-ideal properties and deficiencies in the interferometer result in a reduction in the visibility. Third information which can be defined by way of the visibility and generated by this type of measurement, is referred to as dark field. The dark field specifies the ratio from the visibilities of the measurement with object and those without object.

$$D = \frac{{V}_{obj}}{{V}_{ref}} = \frac{{A}_{obj} \cdot {\overline{I}}_{ref}}{{A}_{ref} \cdot {\overline{I}}_{obj}}$$

Three different images can then be generated from the comparison of specifically derived variables from the fitted functions for each pixel once with and once without an object (or patient).

i) absorption image,

ii) differential phase contrast image (DPC) and

iii) dark field image

If reference is made to an image below, the triumvirate comprising absorption, DPC and dark field image is meant if applicable.

The realization of the method represents many challenges. One of these challenges resides in the very high demand placed on the temporal constancy of the geometric arrangement of the various gratings G₀, G₁ and G₂, since each relative movement of the grating with respect to one another results in phase shifts and thus in local changes to the intensity distributions at the detector input. The method with a Talbot Lau interferometer arrangement is however based on measurements with and without an object, i.e. phase information is used, which was generated at different times and in some instances at different geometric alignments of the x-ray system. The accuracy requirements in the direction of the direction of movement of the analyzer grating G₂ amounts for instance to a fraction of a typical phase step, in other words in the sub μm range. Relative changes in distance between the components required for image generation into the other location directions or also tilts, rotations etc. can also result in the imaging being faulty or even breaking down entirely.

For medical applications, in which highly precise optical banks cannot be used, and here in particular for potential applications in angiography or surgery, whereby x-ray systems with C-arms are used, such as were explained for instance with the aid of the FIG. 1, and are frequently repositioned, in order to enable other angulations or also CT-similar imaging (wedge-beam CT) by rotating the C-arm about the relevant organ or body part, constantly changing forces (gravitation force, centrifugal forces etc.) act on the entire mechanics and the corresponding components, so that conventional, currently used mechanics are not sufficient, since inaccuracies of up to several hundred μm or more may exist.

Other influences may influence the relative geometric arrangement in particular of the gratings G₀, G₁ and G₂ with respect to one another such as for instance temperature changes, vibrations, shocks, mechanical stresses of another type, etc.

Influences of this type can, as described above, result in deviations from the geometry target, in other words a deviation in the position, rotation, tilt etc. of the mechanical units (in particular the grating), relevant to the imaging, relative to one another.

US 2012/250823 A1 associates at least one of the gratings with at least two actuators. These actuators are used to realize the phase shift/phase stepping (in other words the central part of the phase imaging).

Furthermore, US 2012/0260823 A1 describes a calibration with a measurement of the phase contrast without object (first plurality of measurements). This is a central integral part of the “differential” phase contrast imaging when using comparatively poor locally resolving x-ray detectors. Phase contrast images without an object are thus produced (“calibration”) and then with an object (measurement with object). However, this means that for this type of imaging, the “undisturbed” case (no object/calibration: first plurality of measurements) and the “disturbed” case (object in the radiation path: second plurality of measurements) is inherently required in order to obtain the shift in the phase in the case of an object in the radiation path compared with the phase with no object in the radiation path.

GB 1 348 640 discloses an optical measuring system, in which overlays of the waves, in other words interferences, occur by overlaying the original beam and partially reflected beam on a moving object (prism). A relative movement can be measured by measuring the interference pattern (overlay or cancellation of the optical waves or all states therebetween).

U.S. Pat. No. 5,812,629 describes an interferrometric alignment system.

### SUMMARY

At least one embodiment of the invention is directed to an x-ray recording system such that a real-time capable phase contrast imaging is enabled with various loads and alignments of the system components.

An x-ray recording system is disclosed. Advantageous embodiments are specified in the dependent claims.

An x-ray recording system of at least one embodiment includes at least one measuring apparatus for determining deviations in the geometric ratios of the components relative to one another from the geometry target, an analysis unit for evaluating the measured deviations, a computing unit for determining correction values and a correction device for adjusting the geometric ratios of the components.

### BRIEF DESCRIPTION OF THE DRAWINGS

The invention is described below in more detail with reference to exemplary embodiments shown in the drawing, in which;

FIG. 1 shows a known x-ray C-arm angiography system of an interventional suite with an industrial robot as a support apparatus,

FIG. 2 shows a schematic structure of a known Talbot Lau interferometer for the differential phase contrast imaging with extended tube focus, three gratings G₀, G₁ and G₂ and a pixelated detector,

FIG. 3 shows a schematic representation of a set-up for measuring a linear relative movement between two gratings G₀ and G₁,

FIG. 4 shows a schematic representation of a structure for measuring a relative tilt between two gratings G₀ and G₁,

FIG. 5 shows a schematic representation of a control loop for compensating for relative movements between components,

FIG. 6 shows the set-up according to FIG. 3 with the analysis and correction units and an actuator for compensating for a translational movement and

FIG. 7 shows a schematic set-up of an angiography C-arm x-ray recording system with “open geometry” having devices for the optical measurement of relative changes in positions of various components which are relevant to the imaging.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

An x-ray recording system of at least one embodiment includes at least one measuring apparatus for determining deviations in the geometric ratios of the components relative to one another from the geometry target, an analysis unit for evaluating the measured deviations, a computing unit for determining correction values and a correction device for adjusting the geometric ratios of the components.

An x-ray recording system of at least one embodiment, for differential phase contrast imaging of an examination object with phase-stepping, allows for a real-time measurement and correction so as to ensure the required geometric precision in the case of differential phase contrast imaging for medical applications.

It has proven advantageous if the x-ray emitter for generating quasi coherent x-ray radiation has an absorption grating G₀.

The x-ray emitter for generating quasi coherent x-ray radiation can advantageously comprise a plurality of field emission x-ray sources or a sufficiently powerful microfocus source.

In accordance with at least one embodiment of the invention, the measuring apparatus can include optoelectric distance sensors for measuring distances and alignments of the components critical to the phase contrast imaging.

It has proven advantageous if the measuring apparatus has a laser beam source and a photosensor on one side of the C-arm, a mirror arrangement which can be changed in terms of properties on the other side of the C-arm and an optical transmission path.

The isocenter of the x-ray recording system can remain free if the optical transmission path has a folded radiation path adjusted to the x-ray recording system.

A tilt can be detected in accordance with at least one embodiment of the invention if the mirror arrangement which can be changed in terms of its properties has a mirror which can be tilted as a function of the alignment of a component, said mirror deflecting a reflected laser beam across a photodiode array.

A translational linear relative movement can be identified if the mirror arrangement which can be changed in terms of its properties has a rear-sided mirror attached to the rear of a semi-transparent wedge, said mirror attenuating a reflected laser beam differently as a function of the deflection of a component.

The deviations in the geometric ratios of the components relative to one another from the geometry target which are detected by the measuring apparatus another may advantageously be deviations in the position, rotation and/or tilt of the components.

For correction (compliance, reproduction) of the geometry target of the given relative geometric arrangement, the at least one correction device for adjusting the geometric ratios of the components critical to the phase contrast imaging may be inventive actuators.

The actuators for correction may advantageously be piezoactuators and/or stepper motors.

It has proven advantageous for the x-ray image detector to be an integrating detector with indirect conversion of the x-ray quanta by way of Csi as detector material and CMOS for the photodiode and read-out structure or to be implemented as a photon-counting detector with indirect conversion of the x-ray quanta.

FIG. 3 reproduces the principle of detecting a height adjustment of the components in the example of both gratings G₀ and G₁. The absorption grating 13 and the phase grating 17 are held by way of connections 20. The absorption grating 13 is assigned to a laser 21 and a photosensor, for instance a photodiode 22. The laser 21 sends a laser beam 23, which strikes a semitransparent wedge 24 assigned to the phase grating 17 and penetrates the same in an attenuated manner. A rear-sided mirror 24 is attached to the rear of the semitransparent wedge 24, said mirror throwing back a reflected laser beam 26 through the semitransparent wedge 24 in a further attenuated fashion onto the photodiode 22. The reflected laser beam 26 is attenuated relative to the emitted laser beam 23 as a function of the relative movements 27 by vibrations for instance, so that the output signal of the photodiode 22 reproduces the degree of deviation of the position of the phase grating 17.

FIG. 4 illustrates the principle of detecting a tilt of the components in the example of both gratings G₀ and G₁. Instead of the semitransparent wedge 24 and the rear-sided mirror 25, a mirror 28 is attached to the connection 20 of the phase grating 17. The photodiode 22 is replaced by a locally resolved photodiode array 29. By tilting 30 the phase grating 17, the laser beam 26 reflected by the mirror 28 is deflected according to a deflection 31. This measure then specifies the degree of the tilt and can be used for correction purposes.

FIG. 5 now shows a schematic representation of a possible correction arrangement in the form of a control loop between optical measurements of relative movements of the components, evaluation of the measurements, determination of correaction values and use of correction values with the aid of actuators to compensate for relative movements of this type.

A number of components 32 K₁ to Kₙ are influenced in respect of their geometric dimensions and arrangements by effects or influences 33, such as forces, vibrations, movements, shocks, changes in temperature etc., the extents of which are detected by a measuring apparatus 34. These may be the arrangements described with the aid of FIGS. 3 and 4 for instance. The measuring results of the measuring apparatus 24 are supplied to an evaluation apparatus 35, which is connected to a computing unit 36 for determining correction values. The correction values derived from the evaluated measuring results are supplied to a control apparatus 37, which actuates and influences the individual actuators 38 A₁ to Aₙ assigned to the components 32 K₁ to Kₙ so that the detected deviation from the normal is balanced out and corrected.

FIG. 6 shows an arrangement for detecting a height adjustment of the components G₁ according to FIG. 3, however additionally again with the analysis and correction units and also an actuator for compensating for a translational movement along the grating structures of gratings G which are at right angles to the orientation, said gratings being detected in this case by the optical measuring system 20 to 26.

An analysis unit 40 is connected to the photodiode 22, which evaluates the attenuation through the semitransparent wedge 24 of the laser beam 26 reflected by the rear mirror 25 on account of the relative movements 27. The output signal of the analysis unit 40 is supplied to a correction unit 41. This correction unit 41 is connected with a piezoactuator 42 for control thereof, which acts on the phase grating 17 by way of the semitransparent wedge 24 and the connection 20 such that the relative movements 27 are compensated.

FIG. 7 shows an inventive angiographic x-ray recording system of an embodiment with the C-arm 2, the x-ray emitter 3 and the x-ray image detector 4 and the table plate 5 of the patient support couch and the patient 6 to be examined resting thereupon in a schematic representation which is not true to scale. The laser 21 and the photodiode array 29 are attached to the C-arm 2 in the vicinity of the x-ray emitter 3 by way of a holder 43. The laser 21 supports the absorption grating 13 (G₀) by way of the connection 20.

The photodiode array 29 is location-sensitive. Current CCD or CMOS sensors can be used, such as are used in cameras and/or mobile telephones. They have pixel sizes of 1-2 μm and a corresponding resolution for instance.

The phase grating 17 (G₁) and the analyzer grating 18 (G₂) with their connections 20 via a hinge 44 to the C-arm 2 are attached on the opposite side of the C-arm 2 adjacent to the x-ray image detector 4. A piezoactuator 46 is fastened to a mechanical suspension 45, which aligns the arrangement with the two gratings 17 and 18 via the semitransparent wedge 24 with the rear-sided mirror 25. The phase stepping 19 of the analyzer grating 18 (G₂) is achieved with a phase stepper 47, which is arranged between the connection 20 and the analyzer grating 18.

The laser beam 48 originating from the laser 21 is deflected by way of a mirror 49 in a folded radiation path 50 and is guided to the semi-transparent wedge 24. It is reflected there on the rear-sided mirror 25, and fed back in the folded radiation path 50, where it strikes a semitransparent mirror 51 briefly before the first mirror 49, which deflects it as a radiation divider onto the photodiode array 29, where its target deviations are recorded and subsequently evaluated. On account of this evaluation by the analysis 40 and correction unit 41 (not shown in this Figure), the piezoactuator 46 is then actuated, which causes a deflection of the grating arrangement suspended via the hinge 44, so that an unwanted movement can be corrected for instance by “bending” the C-arm 2 in accordance with the invention with the piezoactuator 46 for instance.

This arrangement with the folded radiation path 50 allows for an “open geometry”, such as with currently conventionally used C-arm 2, and consequently the inventive optical measurement of relative changes in positions can be implemented differently for the imaging of relevant components.

By way of the inventive arrangement of an embodiment, a real-time measurement and correction facilities are obtained so as to ensure the necessary geometric precision with differential phase contrast imaging of an examination object with phase-stepping.

In order to measure and correct deviations of this type from the geometry target
optoelectric distance sensors for measuring distances and alignments of the components critical to the phase contrast imaging (in particular the grating),
an analysis unit for evaluating the deviations,
a computing unit for determining correction values and
actuators for the correction (compliance, reproduction) of the geometry target of a given relative geometric arrangement of the components critical of the phase contrast imaging (in particular the grating) are used.

Optoelectric distance sensors include here a transmitter, the light source, a receiver, the detector and an analysis unit. For instance lasers (e.g. laser diodes) of various wave lengths (e.g. red, green, blue) can be used as transmitters and photodiodes, CCD or CMOS sensors or position-sensitive semiconductors as receivers for instance. Since in general no absolute distance measurement is required, but only relative distance changes accompany the various interferences or influences, only changes in certain parameters such as location change in the laser beam, amplitude change, frequency change or polarization change in the laser light are necessary.

As optical methods, delay time or triangulation measurement methods can be used for instance or also interferometric methods.

Mirrors or mirroring surfaces or partial surfaces can if necessary be attached to the relevant units.

Methods can however also be used, which measure the intensity change or the angular deflection of a laser beam, in order to detect translational movements or tilts relative to one another, such as was described for instance with the aid of FIGS. 3 and 4, wherein these figures are only to be considered as simple examples of translational movements or tilts between the gratings G₀ and G₁ for instance.

The subject matter of the present patent application is not to describe a comprehensive collection of such or similar methods, but instead to specify the basic use of such measurement, analysis and correction units, without which, phase contrast imaging without the use of highly precise optical banks with laboratory-style structures would not be possible, and thus not be useable or only be of limited use for medical imaging in a clinical environment.

Piezoactuators, stepper motors or suchlike can be used as actuators for instance.

In order to be able to realize a C-arm-like set-up, a number of mirrors must be provided in some instances, which implement the optical measurements and at the same time enable an “open” region in the C-arm for the patient 6.

In order to measure and correct various relative position or orientation changes between the critical imaging components 32, a number of optical systems, a number of actuators 38 and more complex mechanical suspensions and fastenings are required, such as are symbolized for instance by the connection arrows of the components 32 with the measuring apparatus 34.

At least one embodiment of the inventive arrangement has at least one of the following advantages:
At least one embodiment of the inventive concept enables use of phase contrast imaging outside of a technical facility with optical banks too, in other words for instance in a clinical environment with current usual mechanical components.
At least one embodiment of the concept allows for “open geometries”, such as for instance realized currently in C-arms, whereby no hardware is arranged in the isocenter in which the patient is disposed.

FIG. 3 therefore reproduces a schematic representation of a set-up for measuring the relative movement between e.g. grating G₀ and grating G₁. The optical set-up includes a laser beam source, the laser 21 and/or the laser diode, the photo sensor, such as for instance photodiode 22, photo cell and/or photo diode array 29 (CCD, CMOS), and the semitransparent wedge 24 with a rear-sided mirror 25. The grating G₀ is connected to the laser 21 and the photodiode 22 and the grating G₁ is connected to the semitransparent wedge 24. Depending on the position, more or less light is absorbed through the semitransparent wedge 24, so that an intensity change can be measured on the photo diode 22 or with a locally resolved photo diode array 29 (CCD, CMOS) a variation in the measured intensities in the pixel elements.

With the schematic representation of a set-up for measuring a relative tilt of grating G₁ compared with grating G₀ in FIG. 4, a position-sensitive photodiode array 29 is used, e.g. a CCD or CMOS sensor.

The control loop according to FIG. 5 shows the relationship between optical measurements of relative movements 27, 30 of the components on account of effects or influences 33, such as forces, vibrations, movements, impacts, changes in temperature etc. The evaluation of measurements, determination of correction values and use of correction values with the aid of actuators 38 to compensate for relative movements 27, 30 of this type.

The set-up according to FIG. 6 equates to that in FIG. 3, wherein, however, the analysis 40 and correction units 41 and also the actuator 42 for compensating for the relative movements 27, 30, which can be detected by the optical measuring system in this case, namely the translational relative movement 27 is at right angles to the orientation of the grating structures of the grating G₁.

FIG. 7 shows a schematic set-up of at least one embodiment of an inventive angiographic C-arm x-ray recording system, which allows for an “open geometry” as with currently used C-arms 2, and nevertheless allows for an optical measurement of relative changes in positions of various components 3, 4 which are relevant to the imaging. The analysis 40 and correaction units 41 are not shown, similarly other parts of an x-ray system. The gratings G₁ and G₂ are suspended here via the hinge 44 such that a movement can be corrected with the piezoactuator 46 by “bending” the C-arm 2.

Naturally a number of such laser/sensor-array/actuator combinations can be provided, since the relative movements 27 and/or 30 can comprise different directions/orientations. The relative movements may be for instance a tilting movement 30 according to FIG. 7 or also a linear movement 27 along a rail, air cushion attachment or suchlike.

Optical waveguides, optical fibers, plastic fibers or suchlike can be used at suitable points or under suitable conditions along the optical path in addition to mirrors as part of the optical path or the folded radiation path 50.

The idea can essentially also be used for configurations, in which the grating G₂ is omitted and the phase contrast imaging is realized by way of other methods (e.g. electronic phase stepping). It should also be ensured in such structures that the critical components are aligned geometrically precisely relative to one another during the imaging process.

What is claimed is:

1. An x-ray recording system for differential phase contrast imaging of an examination object by way of phase stepping, comprising:
at least one x-ray emitter, configured to generate quasi coherent x-ray radiation;
an x-ray image detector including pixels arranged in a matrix;
a defraction or phase grating, arranged between the examination object and the x-ray image detector;
an analyzer grating assigned to the defraction or phase grating, wherein x-ray emitter, x-ray image detector, defraction or phase grating and analyzer grating form components critical to the phase contrast imaging in an arrangement;
at least one measuring apparatus configured to determine deviations in the geometric rations of the components relative to one another from the geometry target;
an analysis unit configured to evaluate the measured deviations;
a computing unit configured to determine correction values; and
at least one correction device, configured to set the geometric ratios of the components.

2. The x-ray recording system of claim 1, wherein the x-ray emitter includes an absorption grating for generating quasi coherent x-ray radiation.

3. The x-ray recording system of claim 1, wherein the x-ray emitter for generating quasi coherent x-ray radiation includes a plurality of field emission x-ray sources.

4. The x-ray recording system of claim 1, wherein the x-ray emitter for generating quasi coherent x-ray radiation includes a sufficiently powerful microfocus source.

5. The x-ray recording system of claim 1, wherein the at least one measuring apparatus includes optoelectric distance sensors for measuring distances and alignments of the components critical to the phase contrast imaging.

6. The x-ray recording system of claim 1, wherein the at least one measuring apparatus includes a laser beam source and a photosensor on one side of the C-arm, a mirror arrangement, changeable in terms of its properties on another side of the C-arm, and an optical transmission path.

7. The x-ray recording system of claim 6, wherein the optical transmission path includes a folded radiation path adjusted to the x-ray recording system.

8. The x-ray recording system of claim 6, wherein the mirror arrangement, changeable in terms of its properties, includes a mirror, tiltable as a function of the alignment of a component, said mirror being configured to deflect a reflected laser beam by way of a photo diode array.

9. The x-ray recording system of claim 6, wherein the mirror arrangement, changeable in terms of its properties, includes a rear-sided mirror attached to the rear of a semitransparent wedge, said mirror being configured to attenuate a reflected laser beam differently as a function of the deflection of a component.

10. The x-ray recording system of claim 1, wherein the deviations in the geometric ratios of the components relative to one another from the geometry target, detected by the measuring apparatus, are deviations in at least one of position, rotation and tilt of the components.

11. The x-ray recording system of claim 1, wherein the at least one correction device, for setting the geometric ratios of the components which are critical to the phase contrast imaging, is at least one actuator.

12. The x-ray recording system of claim 11, wherein the at least one actuator is at least one of at least one piezoactuator and at least one stepper motor.

13. The x-ray recording system of claim 1, wherein the x-ray image detector is an integrating detector with indirect conversion of the x-ray quanta by way of CsI as a detector material and CMOS for photodiode and read-out structure.

14. The x-ray recording system of claim 1, wherein the x-ray image detector is implemented as a photon-counting detector with direct conversion of the x-ray quanta.

15. The x-ray recording system of claim 2, wherein the x-ray emitter for generating quasi coherent x-ray radiation includes a plurality of field emission x-ray sources.

16. The x-ray recording system of claim 2, wherein the x-ray emitter for generating quasi coherent x-ray radiation includes a sufficiently powerful microfocus source.

17. The x-ray recording system of claim 2, wherein the at least one measuring apparatus includes optoelectric distance sensors for measuring distances and alignments of the components critical to the phase contrast imaging.

18. The x-ray recording system of claim 2, wherein the at least one measuring apparatus includes a laser beam source and a photosensor on one side of the C-arm, a mirror arrangement, changeable in terms of its properties on another side of the C-arm, and an optical transmission path.

19. The x-ray recording system of claim 2, wherein the x-ray image detector is an integrating detector with indirect conversion of the x-ray quanta by way of CsI as a detector material and CMOS for photodiode and read-out structure.

20. The x-ray recording system of claim 2, wherein the x-ray image detector is implemented as a photon-counting detector with direct conversion of the x-ray quanta.
